(19) United States
(12) Patent Application Publication
(10) Pub. No.: US 2009/0204129 A1
(43) Pub. Date: Aug. 13, 2009

(54) Bioactive wide-weave mesh

(76) Inventor: Leslie Fronio, Annandale, NJ (US)

Correspondence Address: Tyco Healthcare Group LP, 60 MIDDLETOWN AVENUE, NORTH HAVEN, CT, 06473, US

(21) Appl. No.: 11/886,934

(22) PCT Filed: Mar. 22, 2006
(86) PCT No.: PCT/US2006/010330

§ 371 Date: Jan. 5, 2009

Related U.S. Application Data
(60) Provisional application No. 60/664,134, filed on Mar. 22, 2005.

(51) Int. Cl. A61B 17/03

(52) U.S. Cl. 606/151

(57) ABSTRACT

A wide-weave mesh is disclosed which is coated with a bioactive material to enhance the therapeutic efficacy of the mesh. The mesh may be used for the treatment of hernias, vaginal prolapses and other similar injuries.

### CROSS-REFERENCE TO RELATED APPLICATIONS

This application claims the benefit of U.S. Provisional Patent Application No. 60/664,134 filed Mar. 22, 2005, the entire disclosure of which is hereby incorporated by reference herein.

### BACKGROUND

1. Technical Field

The present disclosure relates to a coated surgical mesh that may be used in the treatment of hernias, uterovaginal prolapses and other related injuries.

2. Background of Related Art

A hernia is basically a defect resulting in the protrusion of part of an organ through the wall of a body cavity within which it is normally contained.

For example, a fairly common and well-known type of hernia is a defect in the lower abdominal wall resulting in a sac that may contain a portion of the intestine protruding through the abdominal wall. This is referred to as an inguinal hernia. A defect in the abdominal wall after surgery is referred to as an incisional hernia. Another type of hernia is a defect in the pelvic floor or other supporting structures resulting in a portion of the uterus, bladder, bowel or other surrounding tissue protruding through, e.g., the vaginal wall. This is usually referred to as a uterovaginal prolapse.

A common way of treating hernias is to repair the defect by sutures, whether or not the hernial sac is also sutured or repaired, in order that the protruding organ is contained in its normal position. As the defect generally comprises a weakening and attenuation leading to parting of tissues in a fascial wall, it is usually necessary to apply tension to the sutures in order to close the parted tissues. Thus, the fascial wall is generally pinched or tensioned around the area of the defect in order to close the parted tissues.

It has been suggested to make use of a surgical implant to overlay or close the weakened and parted tissues without the need to pinch or tension the surrounding tissue of the fascia. Such surgical implants generally comprise meshes and are now widely used in inguinal hernia repair. Meshes may be applied subcutaneously (i.e., under the skin) internally or externally of the abdominal wall and may be either absorbable or nonabsorbable depending on the nature and severity of the particular defect being treated. Meshes may be applied in combination with sutures to hold the mesh in place or, alternatively, with sutures that close the parted tissues as in a “non-mesh” technique.

Meshes are usually applied in open surgical procedures, although they may sometimes be applied in laparoscopic surgical procedures. A typical mesh for an inguinal hernia repair includes woven or knitted polypropylene, such as MARLEX® or PROLENE®. Such meshes have a number of desirable properties that make them effective for use in hernia repair. For example, they are made of materials that are suitably inert so as to be less likely to cause adverse reactions when implanted in the body. Furthermore, they are mechanically strong, cheap, easily sterilized, and easy to work with.

It has also been suggested to use meshes in the treatment of uterovaginal prolapse. Meshes that have been proposed for use in the repair of uterovaginal prolapse are similar to those that are used for the repair of inguinal hernias.

Conventionally, open procedures have been preferred for the treatment of hernias with meshes, as relatively broad access to the site of the defect is required to suitably implant and secure a mesh by sutures or such like. However, it is desirable to treat hernias, as when carrying out any surgery, with as little trauma to the patient as possible. Thus, the use of minimally invasive techniques has been suggested for the treatment of hernias. Such surgical techniques have not been considered to be useful in the treatment of uterovaginal prolapse with a mesh, as it has not been considered practical to position a mesh subcutaneously in the vaginal wall due to the difficulty in gaining direct access to this area.

In addition, one disadvantage of currently available meshes used in hernia repair is that they have jagged or rough edges. Thus, improvements to surgical implants such as meshes used to treat hernias and prolapses remain desirable.

### SUMMARY

The present disclosure pertains to a novel wide-weave mesh coated with a bioactive coating to enhance the therapeutic efficacy of the mesh. The mesh is made from filaments which are woven into strands that have spaces between the strands of from about 1 mm to about 10 mm and a maximum residual mass density of about 5 g/m² to about 50 g/m².

The disclosure further describes the use of a wide-weave mesh with a bioactive coating in combination with one or more surgical fasteners.

### BRIEF DESCRIPTION OF THE DRAWINGS

FIG. 1 is an illustration of a hernia.

FIG. 2 is an illustration of the hernia of FIG. 1 when intra-abdominal pressure is raised.

FIG. 3 is an illustration of the hernia of FIG. 1 after repair in accordance with the prior art.

FIG. 4 is an illustration of the hernia of FIG. 1 after an alternate repair in accordance with the prior art.

FIG. 5 is a schematic illustration of the female human vaginal area.

FIG. 6 is a cross-sectional view of the female human vaginal area along the line A-A of FIG. 5

FIGS. 7A and 7B illustrate wide-weave meshes according to the present disclosure having a first shape.

FIGS. 8A, 8B, 8C and 8D illustrate wide-weave meshes according to the present disclosure having a second shape.

FIGS. 9A, 9B, 9C and 9D illustrate wide-weave meshes according to the present disclosure having a third shape.

FIGS. 10A, 10B, 10C and 10D illustrate portions of wide-weave meshes according to the present disclosure attached to a fastening device.

FIG. 11 depicts a perspective view of a fastener which may be used to attach a wide-weave mesh of the present disclosure to tissue, illustrating a side view of a helical fastener.

FIG. 11A depicts another perspective view of a fastener which may be used to attach a wide-weave mesh of the present disclosure to tissue, illustrating an end view of the helical fastener.

FIG. 11B depicts a schematic view of a fastener which may be used to attach a wide-weave mesh of the present disclosure to tissue, illustrating a substantially collapsed helical fastener with a relatively small gap that has been partially inserted into tissue.

FIG. 11C depicts a schematic view of a fastener which may be used to attach a wide-weave mesh of the present disclosure to tissue, illustrating the helical fastener depicted in FIG. 11B completely inserted into tissue.

FIG. 11D depicts a schematic view of a fastener which may be used to attach a wide-weave mesh of the present disclosure to tissue, illustrating a substantially collapsed helical fastener with a relatively large gap that has been partially inserted into the tissue.

FIG. 11E depicts a schematic view of a fastener which may be used to attach a wide-weave mesh of the present disclosure to tissue, illustrating the helical fastener depicted in FIG. 11D completely inserted into tissue.

FIG. 11F depicts a perspective view of another embodiment of a fastener which may be used to attach a wide-weave mesh of the present disclosure to tissue, illustrating an end view of the helical fastener.

FIG. 12 depicts a perspective view of another embodiment of a fastener which may be used to attach a wide-weave mesh of the present disclosure to tissue, illustrating a double helical fastener.

FIG. 12A is a front view of the double helical fastener of FIG. 12.

FIG. 12B is side view of the double helical fastener of FIG. 12.

FIG. 12C is a top view of the double helical fastener of FIG. 12.

FIG. 13 is a perspective view of yet another embodiment of a fastener which may be used to attach a wide-weave mesh of the present disclosure to tissue, illustrating another design of a double helical fastener.

FIG. 13A is a front view of the double helical fastener of FIG. 13.

FIG. 13B is a side view of the double helical fastener of FIG. 13.

FIG. 13C is a top view of the double helical fastener of FIG. 13.

FIG. 14 is a perspective view of another fastener which may be used to attach a wide-weave mesh of the present disclosure to tissue, illustrating a helical fastener with a central post.

FIG. 15 is a perspective view of an absorbable screw fastener which may be used to attach a wide-weave mesh of the present disclosure to tissue.

FIG. 16 is another perspective view of the absorbable screw fastener of FIG. 15.

FIG. 17 is a longitudinal cross-sectional view of the absorbable screw fastener of FIG. 15 taken along line 3-3 of FIG. 15.

FIG. 18 is an orthogonal top view of the absorbable screw fastener of FIG. 17.

### DETAILED DESCRIPTION

According to the present disclosure there is provided a surgical implant suitable for treatment of a hernia, prolapse, or other similar injury. The implant includes a wide-weave mesh having a maximum residual mass density of 50 g/m² or less that is coated with a composition that includes one or more bioactive agents. The residual mass density is the mass density of the mesh after implantation and the absorption of any bioabsorbable coatings. In one embodiment the maximum residual mass density may be less than 30 g/m², while in another useful embodiment the maximum residual mass density may be less than 25 g/m². Thus, in embodiments the maximum residual mass density may be from about 5 g/m² to about 50 g/m², in embodiments from about 15 g/m² to about 40 g/m², in embodiments from about 25 g/m² to about 35 g/m².

The wide-weave mesh of the present disclosure is made of strands which, in turn, may be made of any suitable biocompatible material. Suitable materials from which the mesh can be made should have the following characteristics: sufficient tensile strength to support a fascial wall during repair of a defect in the fascial wall causing a hernia; sufficiently inert to avoid foreign body reactions when retained in the human body for long periods of time; easily sterilized to prevent the introduction of infection when the mesh is implanted in the human body; and have suitably easy handling characteristics for placement in the desired location in the body.

The wide-weave mesh includes strands, major spaces, and pores. The strands of the wide-weave mesh may be formed by at least two filaments, the major spaces formed between the strands providing the surgical implant with the necessary strength, the filaments arranged such that pores are formed in the strands themselves. Alternatively the strands may be formed by monofilaments that are arranged to form loops that give rise to the pores in the strands. The strands may be spaced apart to form major spaces of from about 1 mm to about 10 mm between the strands. In one useful embodiment the strands may be spaced apart to form major spaces of from about 2 mm to about 8 mm between the strands. The use of mesh having strands spaced apart in accordance with the present disclosure has the advantage of reducing the foreign body mass that is implanted in the human body, while maintaining sufficient tensile strength to securely support the defect and tissue being repaired by the wide-weave mesh.

The strands of the wide-weave mesh may have a diameter of less than about 600 μm, in embodiments from about 200 μm to about 600 μm, in embodiments from about 300 μm to about 500 μm. Filaments have a diameter of from about 0.02 mm to about 0.15 mm, in embodiments from about 0.08 mm to about 0.1 mm.

The strands and filaments may be warp knit or woven into a variety of different mesh shapes. In some embodiments the strands may be arranged to form a net mesh which has isotropic or near isotropic tensile strength and elasticity.

Due to the variety of sizes of such defects, and of the various fascia that may need repair by the implant, the implant may be of any suitable size. In one embodiment, the surgical implant is of a width from about 1 cm to about 10 cm and a length from about 1 cm to about 10 cm.

In some embodiments the filaments may be made of a plastic or similar synthetic non-absorbable material. Some examples include polyolefins, such as polyethylene, polypropylene, copolymers of polyethylene and polypropylene, and blends of polyethylene and polypropylene. Polypropylene can be utilized in some embodiments.

In another embodiment the filaments of the mesh may be made of an absorbable material such as a polyester. Some specific examples of suitable absorbable materials which may be utilized to form the filaments include trimethylene carbonate, caprolactone, dioxanone, glycolic acid, lactic acid, glycolide, lactide, homopolymers thereof, copolymers thereof, and combinations thereof.

It can be appreciated that filaments which are made in part of absorbable material would allow better surgical handling and enable the implant to have minimal mass following implantation in the body.

In yet another embodiment, the wide-weave mesh may be made of a material that has memory. A mesh with memory urges the surgical implant to adopt a flat conformation. Such an implant may have a curved perimeter, i.e., few or no corners or apexes, as sharp comers increase the likelihood of edge erosion and infection. The specific shape will, however, vary according to the intended use of the implant.

In embodiments of the present disclosure, filaments may be formed from polypropylene having a diameter of from about 0.07 mm to about 0.08 mm, wherein the strands making up the mesh are spaced to form spaces in the mesh of from about 2 mm to about 5 mm.

In other embodiments, filaments may be formed from polyester having a diameter of from about 0.05 mm to about 0.09 mm, wherein the strands are spaced to form spaces in the mesh of from about 2 mm to about 5 mm.

As the surgical implant includes narrow strands that are spaced by relatively wide gaps, tissue may be slow to grow into the wide-weave mesh of the present disclosure. It thus may be desirable for the mesh to have means for promoting tissue ingrowth. In embodiments, it may be desirable to provide pores in the strands of the mesh to aid tissue ingrowth and to which tissue may more easily adhere.

At least one filament is interwoven or knitted to produce strands having pores which, in turn, are utilized to form a mesh of the present disclosure. For manufacturing reasons, it may be desirable to use two filaments to form pores in the strands of the mesh to assist tissue ingrowth. However, if one filament can be suitably knotted or twisted to form pores of suitable dimensions, this single filament may be used to similar effect to form the strands of the mesh.

The pores of the mesh of the present disclosure are typically of a size that permit fibroblast through-growth and ordered collagen laydown, resulting in integration of the mesh into the body. For example, the woven/knitted filaments create pores in the strands that are from about 50 μm to about 200 μm in diameter, in embodiments from about 55 μm to about 75 μm in diameter. Alternatively, rings or loops of material of from about 50 μm to about 200 μm in diameter may be adhered to or formed on the strands of the mesh to provide additional pores on the strands.

Due to the wide spacing between strands of the mesh of the present disclosure and the small diameter of the filaments, problems found with currently available meshes, i.e., their jagged and/or rough edges, are mitigated.

The wide-weave mesh of the present disclosure possesses a bioactive coating having at least one bioactive agent. The term “bioactive agent”, as used herein, is used in its broadest sense and includes any substance or mixture of substances that have clinical use. Consequently, bioactive agents may or may not have pharmacological activity per se, e.g., a dye. Alternatively, a bioactive agent could be any agent which provides a therapeutic or prophylactic effect; a compound that affects or participates in tissue growth, cell growth and/or cell differentiation; a compound that may be able to invoke a biological action such as an immune response; or a compound that could play any other role in one or more biological processes.

Examples of classes of bioactive agents which may be utilized in accordance with the present disclosure include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of bioactive agents may be used.

Suitable antimicrobial agents which may be included as a bioactive agent in the bioactive coating of the present disclosure include triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B may be included as a bioactive agent in the bioactive coating of the present disclosure.

Other bioactive agents which may be included as a bioactive agent in the composition of the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g. oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included in the bioactive coating of the present disclosure include viruses and cells, peptides, polypeptides and proteins, analogs, muteins, and active fragments thereof, such as immunoglobulins, antibodies, cytokines (e.g. lymphokines, monokines, chemokines), blood clotting factors, hemopoietic factors, interleukins (IL-2, IL-3, IL-4, IL-6), interferons (β-IFN, (α-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, gonadotropins (e.g., FSH, LH, CG, etc.), hormones and hormone analogs (e.g., growth hormone), vaccines (e.g., tumoral, bacterial and viral antigens); somatostatin; antigens; blood coagulation factors; growth factors (e.g., nerve growth factor, insulin-like growth factor); protein inhibitors, protein antagonists, and protein agonists; nucleic acids, such as antisense molecules, DNA and RNA; oligonucleotides; and ribozymes.

A single bioactive agent may be utilized to form the bioactive coating of the wide-weave mesh of the present disclosure or, in alternate embodiments, any combination of bioactive agents may be utilized to form the bioactive coating of the wide-weave mesh of the present disclosure.

A bioactive coating may be applied to the mesh as a composition or coating containing one or more bioactive agents, or bioactive agent(s) dispersed in a suitable biocompatible solvent. Suitable solvents for particular bioactive agents are within the purview of those skilled in the art. In other embodiments, the bioactive coating may include a bioactive agent in a bioabsorbable material.

Absorbable materials which may be combined with a bioactive agent and utilized to form the bioactive coating of the present disclosure include soluble hydrogels such as gelatin or a starch, or cellulose-based hydrogels. In embodiments, the absorbable material may be an alginate or hyaluronic acid. Other examples of absorbable materials which may be utilized to form the bioactive coating include trimethylene carbonate, caprolactone, dioxanone, glycolic acid, lactic acid, glycolide, lactide, homopolymers thereof, copolymers thereof, and combinations thereof. The bioactive coating may have any thickness or bulk and may be utilized to provide the wide-weave mesh with suitable handling characteristics. In embodiments the coating may be in the form of a sheet having a thickness greater than that of the mesh.

In some embodiments, the bioactive coatings of the present disclosure may include a fatty acid component that contains a fatty acid, a fatty acid salt, or a salt of a fatty acid ester. Suitable fatty acids may be saturated or unsaturated, and include higher fatty acids having more than about 12 carbon atoms. Suitable saturated fatty acids include, for example, stearic acid, palmitic acid, myristic acid and lauric acid. Suitable unsaturated fatty acids include oleic acid, linoleic acid, and linolenic acid. In addition, an ester of fatty acids, such as sorbitan tristearate or hydrogenated castor oil, may be used.

Suitable fatty acid salts include the polyvalent metal ion salts of C₆ and higher fatty acids, particularly those having from about 12 to about 22 carbon atoms, and mixtures thereof. Fatty acid salts including the calcium, magnesium, barium, aluminum, and zinc salts of stearic, palmitic and oleic acids may be useful in some embodiments of the present disclosure. Particularly useful salts include commercial “food grade” calcium stearate which includes a mixture of about one-third C₁₆ and two-thirds C₁₈ fatty acids, with small amounts of the C₁₄ and C₂₂ fatty acids.

Suitable salts of fatty acid esters which may be included in the bioactive coatings of the present disclosure include calcium, magnesium, aluminum, barium, or zinc stearoyl lactylate; calcium, magnesium, aluminum, barium, or zinc palmityl lactylate; calcium, magnesium, aluminum, barium, or zinc olelyl lactylate; with calcium stearoyl-2-lactylate (such as the calcium stearoyl-2-lactylate commercially available under the tradename VERV from American Ingredients Co., Kansas City, Mo.) being useful in some embodiments. Other fatty acid ester salts which may be utilized include lithium stearoyl lactylate, potassium stearoyl lactylate, rubidium stearoyl lactylate, cesium stearoyl lactylate, francium stearoyl lactylate, sodium palmityl lactylate, lithium palmityl lactylate, potassium palmityl lactylate, rubidium palmityl lactylate, cesium palmityl lactylate, francium palmityl lactylate, sodium olelyl lactylate, lithium olelyl lactylate, potassium olelyl lactylate, rubidium olelyl lactylate, cesium olelyl lactylate, and francium olelyl lactylate.

Where utilized, the amount of fatty acid component can be from about 5 percent to about 50 percent by weight of the total bioactive coating, in embodiments from about 10 percent to about 20 percent by weight of the total bioactive coating.

Any coating composition containing the bioactive agent may encapsulate an entire filament, strand or mesh. Alternatively, the bioactive coating may be applied to one or more sides of a filament, strand or mesh. Such a coating will improve the desired therapeutic characteristics of the mesh.

The bioactive coating may be applied to the wide-weave mesh utilizing any suitable method known to those skilled in the art. Some examples include, but are not limited to, spraying, dipping, layering, calendaring, etc. The bioactive agent or bioactive coating may also be incorporated into the absorbable coatings described herein and applied to the wide-weave mesh accordingly.

The bioactive coating may add bulk to the mesh such that it is easier to handle. Where the bioactive coating includes a bioabsorbable material, the coating should be released into the body after implantation and therefore should not contribute to the foreign body mass retained in the body. Thus, the advantages of a surgical implant having minimal mass are retained.

Where the bioactive coating includes an absorbable material, the coating may be released into the body within a period of time from about 2 days to about 14 days following implantation. In one embodiment the coating may be released from about 2 days to about 3 days following implantation. In another useful embodiment, the coating may be released from about 7 days to about 14 days following implantation.

The rate of release of a bioactive agent from the bioactive coating on a mesh of the present disclosure can be controlled by any means within the purview of one skilled in the art. Some examples include, but are not limited to, the depth of the bioactive agent from the surface of the coating; the size of the bioactive agent; the hydrophilicty of the bioactive agent; and the strength of physical and physical-chemical interaction between the bioactive agent, the bioactive coating and/or the mesh material. By properly controlling some of these factors, a controlled release of a bioactive agent from the mesh of the present disclosure can be achieved.

In another embodiment, the wide-weave mesh of the present disclosure may comprise a backing strip which may be releasably attached to the mesh. The backing strip may be formed from a range of materials, including plastics, and may be releasably attached by an adhesive.

The releasable attachment of a backing strip to the mesh may provide a more substantial and less flexible surgical implant, which may be more easily handled by a surgeon. Following suitable placement of the surgical implant, the backing strip can be removed from the surgical implant, the surgical implant being retained in the body and the backing material being removed by the surgeon. The surgical implant can therefore benefit from reduced mass while still providing characteristics required for surgical handling.

In embodiments, filaments used to produce the strands of the wide-weave mesh of the present disclosure may be made of bicomponent microfibers. Bicomponent microfibers may include a core material and a surface material. In embodiments, the bicomponent microfibers may include a nonabsorbable or long lasting absorbable core and a shorter lasting absorbable surface material. The surface material of the bicomponent microfiber may be absorbed by the body within a number of hours, such that only the core portion is left in the body for an extended period of time, typically for a long enough period of time to enable tissue ingrowth. Although a variety of materials may be used in forming these bicomponent microfibers, suitable materials include polypropylene for the core and polylactic acid or polyglycolic acid for the surface material. In another embodiment, the bicomponent microfibers may be made of a core material which may be rapidly absorbed by the body and a surface material which is not absorbed as rapidly, i.e., it is absorbed over a longer period of time than the core.

In embodiments, the surface material of the bicomponent microfibers provides the surgical implant with characteristics required for surgical handling. After insertion in the body, the surface material of the bicomponent microfiber may be absorbed by the body leaving behind the reduced mass of the core material as the strands of the mesh.

It may be desirable to provide a variety of implants having different sizes so that a surgeon can select an implant of suitable size to treat a particular patient. This allows implants to be completely formed before delivery, ensuring that the smooth edge of the implant is properly formed under the control of the manufacturer. The surgeon would thus have a variety of differently sized (and/or shaped) implants to select the appropriate implant to use after assessment of the patient.

In another embodiment the mesh can be cut to any desired size. The cutting may be carried out by a surgeon or nurse under sterile conditions such that the surgeon need not have many differently sized implants on hand, but can simply cut a mesh to the desired size of the implant after assessment of the patient. In other words, the implant may be supplied in a large size and be capable of being cut to a smaller size, as desired.

Even where the cutting of the mesh causes an unfinished edge of the mesh to be produced, this unfinished mesh is not likely to cause the same problems as the rough and jagged edges of the implants of the prior art, due to the fewer strands, smaller diameter filaments and treatment of the mesh with a coating which protects the tissue from the mesh during the surgical procedure when damage is most likely to occur.

Different shapes are suitable for repairing different defects in fascial tissue, and thus by providing a surgical implant which can be cut to a range of shapes, a wide range of defects in fascial tissue can be treated.

More broadly, the present disclosure recognizes that the implant can have any shape that conforms with an anatomical surface of a human or animal body that may be subject to a defect to be repaired by the implant.

Typically an anterior uterovaginal prolapse is elliptical in shape or a truncated ellipse, whereas a posterior prolapse is circular or ovoid in shape. Accordingly, the implant shape may be any one of elliptical or truncated ellipse, round, circular, oval, ovoid or some similar shape to be used depending on the hernia or prolapse to be treated.

In this regard, while the surgical implant of the present disclosure may be useful for the repair of uterovaginal prolapse, it may also be used in a variety of surgical procedures including the repair of hernias.

To further reduce edge problems, the wide-weave mesh of the present disclosure may have a circumferential member which extends, in use, along at least part of the perimeter of the implant to provide a substantially smooth edge. In other words, the mesh may have at least one circumferential member (i.e., fiber, strand or the like) that extends around at least part of its circumference so that at least part of the perimeter of the implant is defined by the circumferential member. Alternatively, at least a part of the perimeter of the implant may be defined by more than one circumferential member, at the edge of the mesh.

The edge of the mesh, and hence the perimeter of the implant, can therefore be generally smooth and thus has significant advantages over conventional surgical meshes. Specifically, an implant having a smooth edge is less likely to cause edge extrusion or erosion.

Any amount of the perimeter of the implant may be defined by the circumferential member(s). In one embodiment, at least 50% of the perimeter of the implant may be defined by the circumferential member(s). In another embodiment, at least 80% of the perimeter of the implant may be defined by the circumferential member(s). In order to maximize the benefits of the mesh of the disclosure, it may be desirable in some embodiments to have 100% of the perimeter of the implant defined by the circumferential member(s). Thus, from about 50% to about 100% of the perimeter of the implant may be defined by the circumferential member(s), in embodiments from about 65% to about 95% of the perimeter of the implant may be defined by the circumferential member(s). The majority or whole of the perimeter of the mesh being smooth minimizes the risk of a rough edge causing edge erosion or infection.

The circumferential member(s) may be arranged in a variety of ways to provide the smooth edge or perimeter of the mesh of the present disclosure. In some cases it may be desirable to minimize the number of members utilized to form the perimeter. This simplifies the construction of the mesh, which is desirable not only for manufacture, but also because simpler structures are less likely to have defects which might be problematic after implantation. In embodiments, the perimeter of the mesh may be defined by one circumferential member.

In another embodiment, the mesh may have a plurality of circumferential members arranged at different radial locations. In order to provide an implant of a desired dimension, the periphery of the mesh outward of the desired circumferential member may be cut away such that one or more selected circumferential members form the perimeter of the implant as desired.

The circumferential members may also be arranged concentrically. A concentric arrangement of a plurality of circumferential members conveniently allows maintenance of the shape of the implant for different sizes of implant and provides the mesh with an even structure.

The circumferential members can also be arranged to join with one another in order to form an integral mesh. Alternatively, the mesh may additionally comprise transverse members which extend across the circumferential members joining the circumferential members.

The transverse members may extend radially from a central point to the perimeter of the implant. The transverse members may be arranged to provide substantially even structural strength and rigidity to the implant.

In some embodiments, it may be desirable to secure the mesh in place once it has been suitably located in the patient. The wide-weave mesh can be secured in any manner known to those skilled in the art. Some examples include suturing the mesh to strong lateral tissue, gluing the mesh in place using a biocompatible glue, or using a surgical fastener, e.g., a tack, to hold the mesh securely in place.

In embodiments it may be advantageous to use a biocompatible glue since it is fairly quick to apply glue to the area around the surgical implant. Additionally, the mesh may include at least one capsule containing a biocompatible glue for securing the implant in place. In certain situations the mesh may include up to about four capsules containing a biocompatible glue which may be provided around the perimeter of the surgical implant. The capsules may be hollow thin-walled spheres from about 3 mm to about 5 mm in diameter and may be made of gelatin.

Any biocompatible glue within the purview of one skilled in the art may be used. In embodiments useful glues include fibrin glues and cyanoacrylate glues.

In another embodiment, the wide-weave mesh of the present disclosure may be secured to tissue using a surgical fastener such as a surgical tack. Other surgical fasteners which may be used are within the purview of one skilled in the art, including staples, clips, helical fasteners, and the like.

In embodiments, it may be advantageous to use surgical tacks as a surgical fastener to secure the wide-weave mesh. Tacks are known to resist larger removal forces compared with other fasteners. In addition, tacks only create one puncture as compared to the multiple punctures created by staples. Tacks can also be used from only one side of the repair site, unlike staples, clips or other fasteners which require access to both sides of the repair site. This may be especially useful in the repair of a vaginal prolapse, where accessing the prolapse is difficult enough without having to access both sides of the prolapse. Suitable tacks which may be utilized to secure the wide-weave mesh of the present disclosure to tissue include, but are not limited to, the tacks described in U.S. Patent Application Publication No. 2004/0204723, the entire disclosure of which is incorporated by reference herein.

Suitable structures for other fasteners which may be utilized in conjunction with the wide-weave mesh of the present disclosure to secure same to tissue are known in the art and can include, for example, the suture anchor disclosed in U.S. Pat. No. 5,964,783 to Grafton et al., the entire disclosure of which is incorporated by reference herein. Additional fasteners which may be utilized and tools for their insertion include the helical fasteners disclosed in U.S. Pat. No. 6,562,051 and the screw fasteners disclosed in International Patent Application No. PCT US04/18702, filed on Jun. 14, 2004, the entire disclosure of each of which are incorporated by reference herein.

The surgical fasteners useful with the wide-weave mesh herein may be made from bioabsorbable materials, non-bioabsorbable materials, and combinations thereof. Suitable materials which may be utilized include those described in U.S. Patent Application Publication No. 2004/0204723 and International Patent Application No. PCT US04/18702, the entire disclosure of each of which are incorporated by reference herein. Examples of absorbable materials which may be utilized include trimethylene carbonate, caprolactone, dioxanone, glycolic acid, lactic acid, glycolide, lactide, homopolymers thereof, copolymers thereof, and combinations thereof Examples of non-absorbable materials which may be utilized include stainless steel, titanium, nickel, chrome alloys, and other biocompatible implantable metals. In embodiments, a shape memory alloy may be utilized as a fastener. Suitable shape memory materials include nitinol.

Surgical fasteners utilized with the wide-weave mesh of the present disclosure may be made into any size or shape to enhance their use depending on the size, shape and type of tissue located at the repair site.

The surgical fasteners, e.g., tacks, may be used alone or in combination with other fastening methods described herein to secure the mesh to the hernia, prolapse, or other repair site. For example, the wide-weave mesh may be tacked and glued, or sutured and tacked, into place.

The surgical fasteners may be attached to the wide-weave mesh in various ways. In embodiments, the ends of the mesh may be directly attached to the fastener(s). In other embodiments, the mesh may be curled around the fastener(s) prior to implantation. In yet another embodiment, the fastener may be placed inside the outer edge of the mesh and implanted in a manner which pinches the mesh up against the fastener and into the site of the injury.

According to another aspect of the present disclosure, there is provided a minimally invasive method of treating uterovaginal prolapse which includes the following steps: making an incision in the vaginal wall close to the opening of the vaginal cavity; making a subcutaneous cut, through the incision, over and surrounding the area of the prolapse, which cut is substantially parallel to the vaginal wall; and inserting a wide-weave mesh according to the present disclosure through the incision, into the space defined by the cut.

Thus, a mesh according to the present disclosure can be inserted through a small incision (e.g., from about 1 cm to about 2 cm in length) in the region of the periphery or opening of the vaginal cavity. An incision in this position is easier for a surgeon to access than an incision deeper in the vaginal cavity. It is also more convenient to treat a vaginal prolapse by implanting a mesh of the present disclosure through such an incision.

In one embodiment, the incision may be at the anterior or posterior extremity of the prolapse sac of the vaginal cavity. This may be desirable, as prolapse most often occurs in the anterior or posterior vaginal wall, so positioning the incision in such a location allows the most convenient access to these parts of the vaginal wall.

Suitable placement of the mesh by minimally invasive techniques, particularly in the treatment of uterovaginal prolapse, requires the mesh to be as flexible as possible. Therefore the bioactive coating on the mesh should be strategically placed to ensure the mesh remains foldable, rollable, flexible, etc. In some embodiments, a flexible, less bulky mesh may be more easily handled in the repair of a prolapse by certain tools. Tools that may be used to carry out this procedure are known to those skilled in the art. An example of a suitable tool is disclosed in PCT Application No. PCT/GB02/01234, the entire disclosure of which is incorporated by reference herein. Any tool capable of properly inserting the mesh may ultimately be used.

Embodiments of the present disclosure will now be described, by way of example only, with reference to the accompanying drawings.

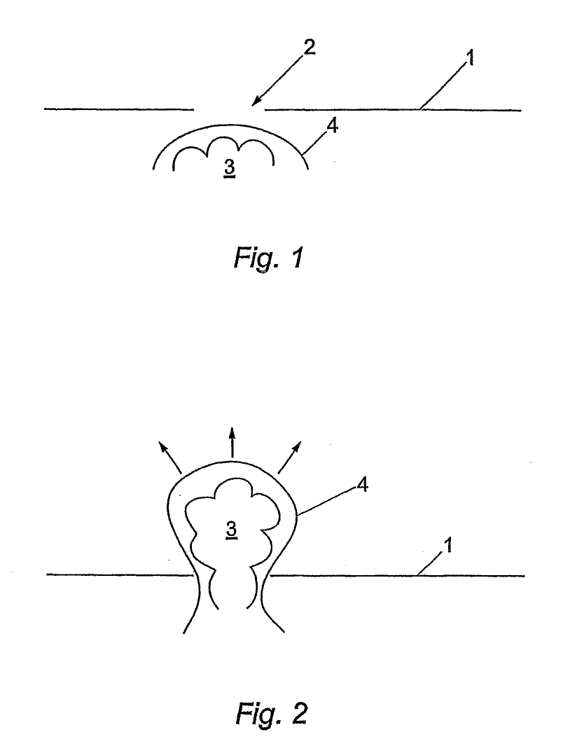

Referring to FIGS. 1 and 2, a hernia, vaginal prolapse or similar injury occurs when a fascial wall 1 ruptures, forming a defect 2, i.e. a weakening or, in this case, parting of the fascial wall 1. An organ 3, contained by the fascial wall 1 is then able to protrude through the defect 2. Such protrusion is illustrated in FIG. 2 and occurs particularly when pressure within the cavity defined by the fascial wall 1 is raised. For example, in the case of an inguinal hernia, when a patient coughs, intra-abdominal pressure is raised and the intestines may be pushed through the defect 2 in the abdominal wall.

While the organ 3 that may protrude through the defect 2 is usually still contained by some other membrane 4, the hernia, prolapse or such like is inevitably painful and liable to infection or other complications. An effective and desirable treatment is therefore to close the defect 2 and contain the organ 3 in its normal position.

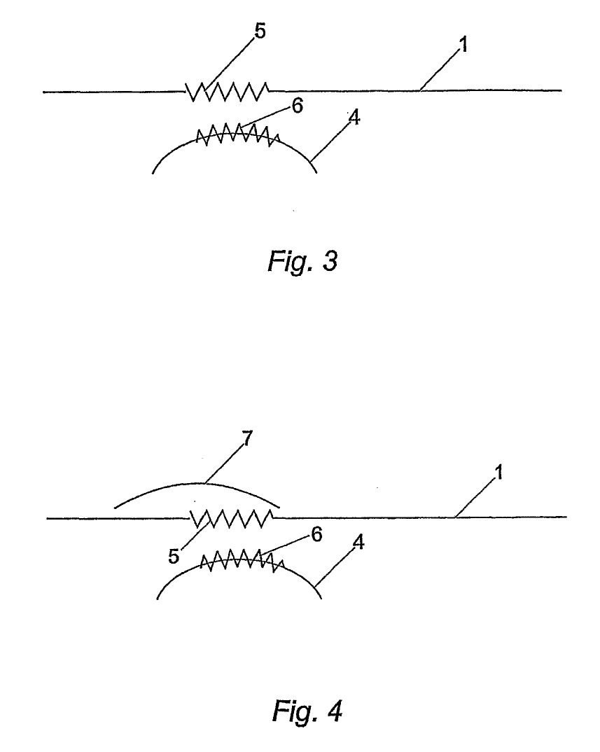

Referring to FIG. 3, a hernia or vaginal prolapse may be conventionally repaired by providing sutures 5 across the defect 2 to join the tissues of the fascial wall 1. In addition, it may be necessary to plicate (i.e. fold or reduce) the other membrane 4 as this may have stretched due to distension of the organ 3. Plication of the other membrane 4 corrects the stretching and helps to relieve pressure on the area of defect 2 during healing as the other membrane 4 can act to contain the organ 3 to some extent. Plication is generally achieved by applying sutures 6 to the other membrane 4.

Referring to FIG. 4, it is also a known method of treating hernias to provide, additionally or alternatively to sutures, a mesh 7 across defect 2. This allows for the defect 2 to be repaired without the parted tissues of the fascial wall 1 necessarily being brought together and for the defect to heal without the fascial wall 1 being pinched or tensioned to correct the defect 2.

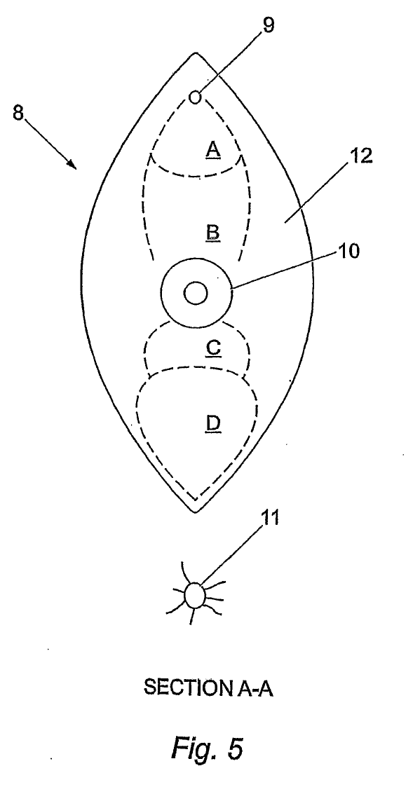

FIG. 5 schematically illustrates (a sagittal view of) the female human vaginal area. The vagina 8 is illustrated with its anterior portion (front) at the top of the diagram and the posterior portion (rear) at the bottom of the diagram. The opening of the urethra, or urethral meatus, 9 is at the forward or anterior end of the vagina 8. The central portion of the vagina 8 forms the vaginal cavity which terminates at the cervix 10. Spaced from the rearward or posterior end of the vagina 8 is the anus 11. Four areas A to D of the vaginal wall 12 are outlined in FIG. 5. These areas A to D are those areas of the vaginal wall 12 in which vaginal prolapse often occurs.

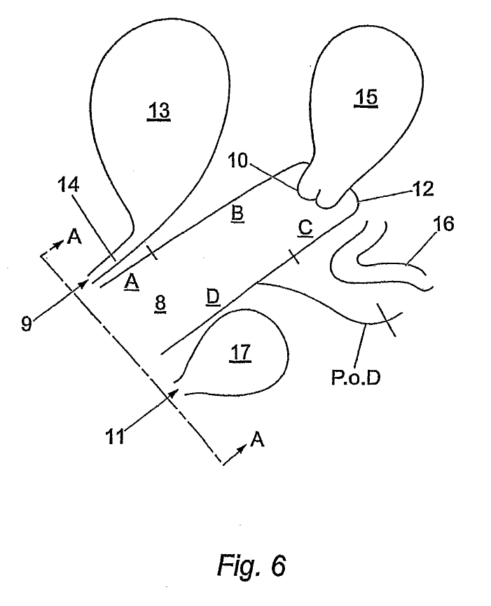

Referring to FIG. 6, which is a cross sectional view along the line A-A in FIG. 5, it can be more clearly seen that the wall 12 of the vagina 8 is bounded by the bladder 13 and urethra 14, the uterus 15, the small bowel 16 and rectum 17. The small bowel 16 and rectum 17 are separated by the Pouch of Douglas.

Area A is the lower one third of the anterior vaginal wall 12 (i.e. the one third nearest the entrance to the vaginal cavity) adjacent the bladder 13 and urethra 14. Prolapse in this area is referred to as anterior or, more specifically, urethracoele prolapse. Area B is the upper two thirds of the anterior vaginal wall 12. Prolapse in this area is referred to as anterior or, more specifically, cystocoele prolapse. The central area of the vaginal wall 12 in which the cervix 10 is located is adjacent the uterus 15 and prolapse in this area is referred to as central, uterine or vault prolapse. Area C is the upper one third of the posterior vaginal wall 12. This area of the vaginal wall 12 is adjacent the small bowel 16 and prolapse in this area is referred to as posterior or enterocoele prolapse. Finally, area D is the lower two thirds of the posterior vaginal wall and is adjacent the rectum 17. Prolapse in this area is generally referred to as posterior or rectocoele prolapse.

Conventionally, any of the above types of hernia have been treated by providing sutures in the area of the prolapse. For example, the extent of the defect causing the prolapse is first identified by the surgeon. Lateral sutures, i.e. sutures from one side to the other of the vaginal wall 12 as seen in FIG. 5, or right to left rather than anterior to posterior, are provided across the area of the defect. This joins the parted tissues of the vaginal wall and repairs the defect. The organ protruding through the vaginal wall is therefore contained. Disadvantages of this technique include anatomical distortion of the vagina due to tensioning of the wall by the sutures to repair the defect.

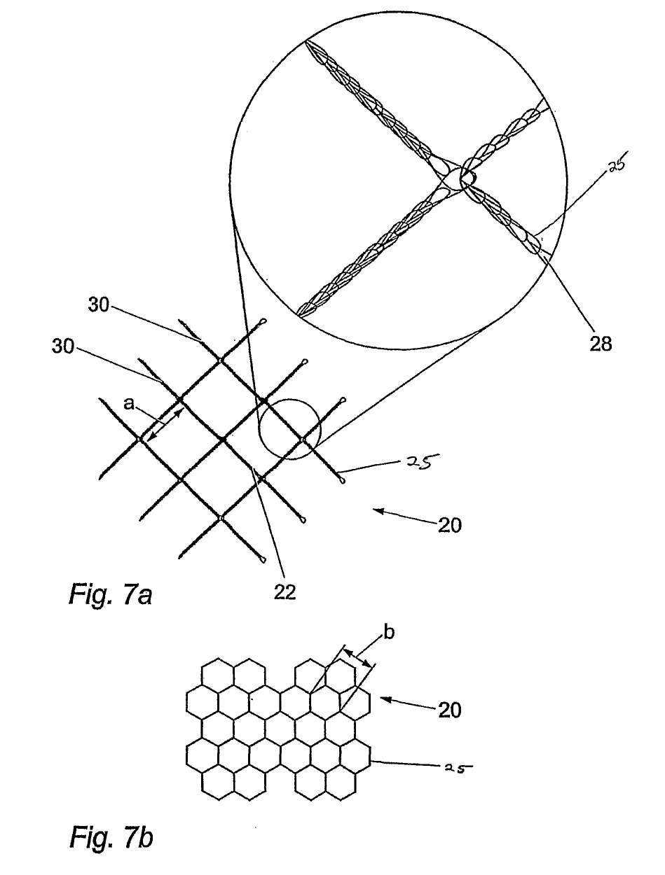

Turning FIGS. 7A and 7B, a surgical implant for use in the repair of vaginal prolapse in accordance with an embodiment of the present disclosure comprises a coated mesh 20. The mesh is comprised of strands 22. The strands may be less than about 600 μm, and approximately from about 150 μm to about 600 μm in diameter. The strands are arranged such that they form a regular network and are spaced apart from each other such that, for a diamond shaped mesh, a space of from about 2 mm to about 5 mm exists between the points where the strands of the mesh interact with each other as depicted in FIG. 7A. In a hexagonal net arrangement, the space is from about 2 mm to about 5 mm between opposite diagonal points where the strands of the mesh interact as depicted in FIG. 7B.

It may be desirable to space the strands as far as part as possible to allow blood to pass through the implant and reduce the mass of the implant, while providing the mesh with sufficient tensile strength and elasticity to be effective. It can therefore be appreciated that considerable variability in the maximum spacing between the strands can be achieved depending on the material from with the strands are made and the net pattern in which the strands are arranged.

In the embodiment shown in FIG. 7A, the strands are arranged in a diamond net pattern, however any pattern which provides suitable tensile strength and elasticity may be used. For example a hexagonal net pattern may be used as shown in FIG. 7B. Ideally, in order to reduce the overall mass of the implant, the strands 22 should have as narrow a diameter as possible while still providing the mesh 20 with suitable tensile strength and elasticity.

The strands 22 of the mesh 20 may be comprised of at least two filaments 25 arranged to interact such that pores 28 are formed between the filaments 25. The pores 28 formed between the filaments 25 may be from about 50 μm to about 200 μm in the diameter, which permits fibroblast through-growth to occur. This fibroblast through-growth secures the implant 20 in place within the body. The suitably sized pores allow the implant 20 to act as a scaffold to encourage the lay down of new tissue. The lay down of new tissue promotes the healing of the hernia or proplapse being treated.

The filaments 25 may be formed from any biocompatible material. In one embodiment the filaments 25 may be formed from polyester, wherein each polyester filament 25 is about 0.09 mm in diameter. In the embodiment shown the filaments 25 of the strands 22 are knitted together using a warp knit to reduce the possibility of fraying of the filaments 25 and strands 22.

The fine warp knit of the filaments 25 provide a surgical implant which is flexible in handing and which can be easily cut into different shapes and dimensions. As the strands 22 are formed using warp knit, the possibility of fraying of the edge of the surgical implant 20 following production or cutting of the surgical implant 20 is reduced.

Other methods of reducing fraying of the filaments are heat treatment, laser treatment or the like, to seal the edges of the surgical implant.

The mesh 20 may be supplied in any shape or size and cut to the appropriate dimensions as required by the surgeon.

It can be appreciated that cutting of the mesh will produce an unfinished edge. Due to the sparse nature of the strands that form the mesh and their narrow diameter, this unfinished edge does not suffer from the same problems as edges of meshes of the prior art. In other words, the edge produced is not rough and jagged such that it increases the likelihood of extrusion of the edge of the mesh in situ or the chance of infection.

As discussed above, an advantage of the mesh of the present disclosure is that it allows the production of a mesh suitable for use in hernia repair which allows substantially less foreign material to be left in the body.

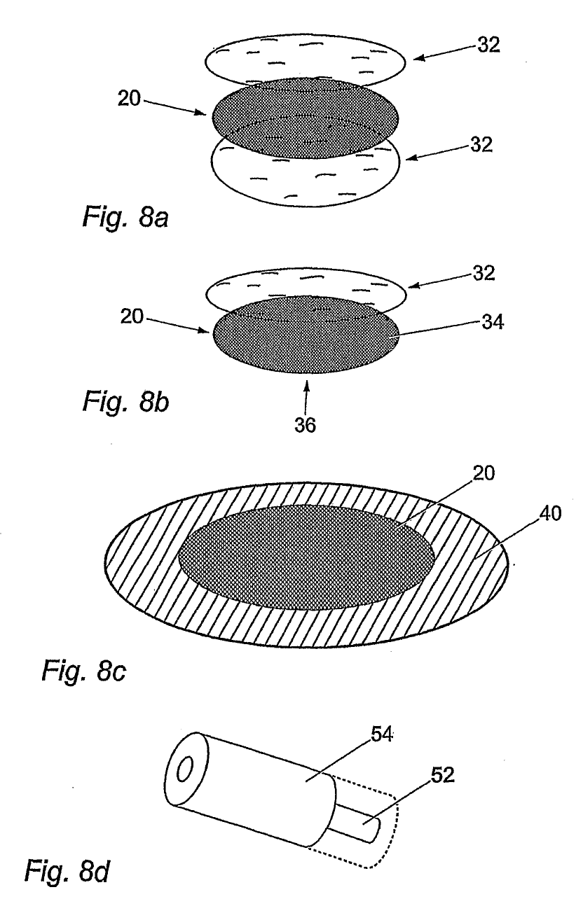

Referring to FIGS. 8A and 8B, the mesh includes a bioactive coating 32. The bioactive coating 32 may, in some embodiments, comprise a layer of absorbable material possessing at least one bioactive agent, wherein the coating layer has a thickness greater than that of the strands 22 of the mesh 20. For example, the thickness of the layer of coating material may be about 1 mm to about 2 mm. The strands of the mesh 20 may be entirely embedded in the bioactive coating 32 such that the outer surface of the mesh 20 is covered entirely by the bioactive coating 32. In effect, the entire surgical implant may be encased in the bioactive coating as shown in FIG. 8A.

Thus, the surgical implant has no gaps or holes on its surface. This has the advantage of reducing the likelihood of bacteria becoming lodged on the strands of the mesh 20 before implantation of the mesh 20. Furthermore, the bioactive coating 32 makes the mesh 20 more substantial and less flexible such that it is more easily handled by a surgeon. This is particularly useful when it is desired to place the mesh in a desired location in a conventional, open surgical procedure.

In an alternate embodiment shown in FIG. 8B, the bioactive coating 32 comprises a layer of coating material applied to one face 34 of the mesh 20, such that the mesh has a first face 34 on which the coating material has been applied and a second face 36 on which the coating material has not been applied. Thus, the first and second faces 34 and 36 each have different characteristics.

In another embodiment depicted in FIG. 8C, a surgical implant may be desired utilizing the releasable attachment of the mesh 20 to a backing strip 40. The backing strip may be formed from a plastic material and may be adhered to the surgical implant using a releasable adhesive. The backing strip 40 causes the mesh 20 to be more substantial and less flexible such that it is more easily handled by a surgeon. Following the suitable placement of the mesh 20, the backing strip 40 can be removed from the mesh 20, the mesh 20 being retained in the body and the backing material 40 being removed by the surgeon. An implant possessing backing strip 40 applied to mesh 20 means the mesh 20 benefits from reduced mass but the mesh 20 and backing strip 40 together may provide desirable characteristics for surgical handling.

As shown in FIG. 8D, in a further embodiment the filaments of the mesh may be comprised from bicomponent microfibers. The bicomponent microfibers may include a core 52 (cutaway section shows core region) and surface material 54. The surface material 54 is designed such that it is absorbed by the body in a matter of hours, while the core material 52 remains in the body for a longer period to enable tissue ingrowth.

Suitable bicomponent microfibers include a polypropylene non-absorbable portion and a polylactic acid absorbable portion. The surface material 54 is present during the surgical procedure when the mesh is being inserted and located in the patient, and provides the mesh with characteristics desirable for surgical handling. Following a period of insertion in the body, typically a few hours, the surface material 54 is absorbed into the body leaving only the core material 52 of the filaments in the body.

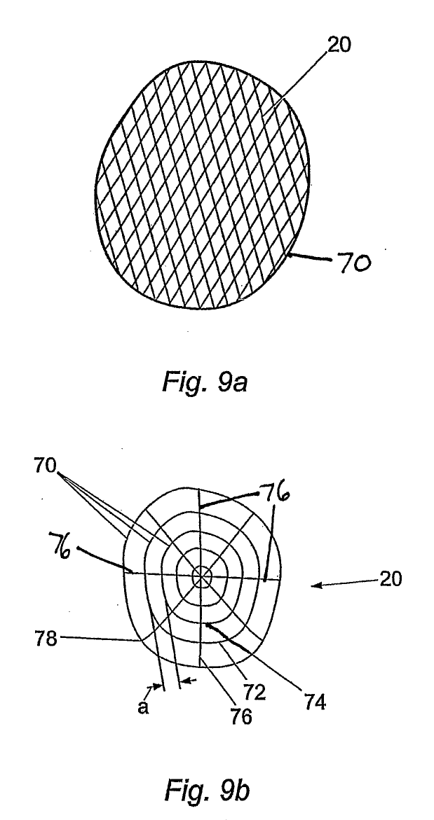

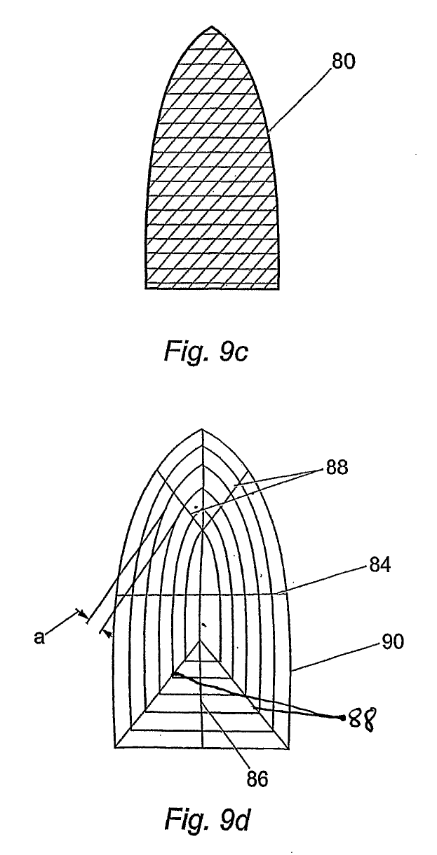

Referring to FIGS. 9A and 9B, a further embodiment of the mesh may include perimeter strands. Typically the mesh 20 is circular or the like in shape and the perimeter strand can be generally referred to as a circumferential strand 70.

In the example shown in FIG. 9A, one strand 70 runs around the circumference of the oval shape of the mesh 20. In another embodiment, several circumferential strands may be present, each circumferential strand extending over one side of the oval mesh, e.g., around half the circumference of the mesh, a quarter of the circumference of the mesh, etc.

As shown in FIG. 9B, the circumferential strands 70 may also be arranged concentrically and each extend around the mesh 20 at a different radial location. An outer circumferential strand 78 extends around the perimeter of the mesh 20, and further circumferential strands 72 and 74 are arranged inwardly of the outer circumferential strand forming a perimeter spaced by a distance (a). The distance (a) between adjacent circumferential members 78, 72 and 74 can vary and, in this example, is about 20 mm.

As also depicted in FIG. 9B, transverse strands 76 may be present which extend from the center of the oval mesh to points on the perimeter of the mesh 78. In this example, four transverse strands 76 are provided across the diameter of the mesh 20, dividing the mesh into eight angularly equal portions.

The mesh 20 of this embodiment may be formed from materials as previously described. Depending on the material chosen, the mesh may be woven, knitted or extruded as one piece, or individual or groups of strands can be extruded separately and joined to one another.

Such a construction as described above provides a mesh 20 with sufficient tensile strength to repair defects causing vaginal prolapse while having minimal bulk. Similarly, such a construction provides a flexible yet resilient mesh for handling.

Referring to FIGS. 9C and 9D, meshes 80 and 90 may be produced having angled sides. These meshes have a similar structure to that described with reference to FIGS. 9A and 9B. Further, the mesh may have transverse members arranged only to extend towards the perimeter of the mesh, rather than all being across the diameter of the mesh. This provides a more uniform structure. More specifically, referring to FIG. 9D, the mesh may have a transverse member 84 extending along its axis of symmetry, a transverse member 86 bisecting the axis of symmetry, and four further transverse members 88 extending from the axis of symmetry to the perimeter of the mesh 90.

In addition to the pores provided by the combination of filaments which form the strands of the mesh, pores can be provided by rings of polypropylene positioned at the intersection of the circumferential and transverse members.

Alternatively, pores may be formed by the spacing of the transverse members, such that pores of a size of from about 50 μm to about 200 μm suitable for enabling tissue ingrowth exist between the transverse members.

To secure the mesh to a suitable location in the body, a number of methods can be used. The bioactive coating may be tacky and thus suitable to hold the mesh in place until it is secured by tissue ingrowth.

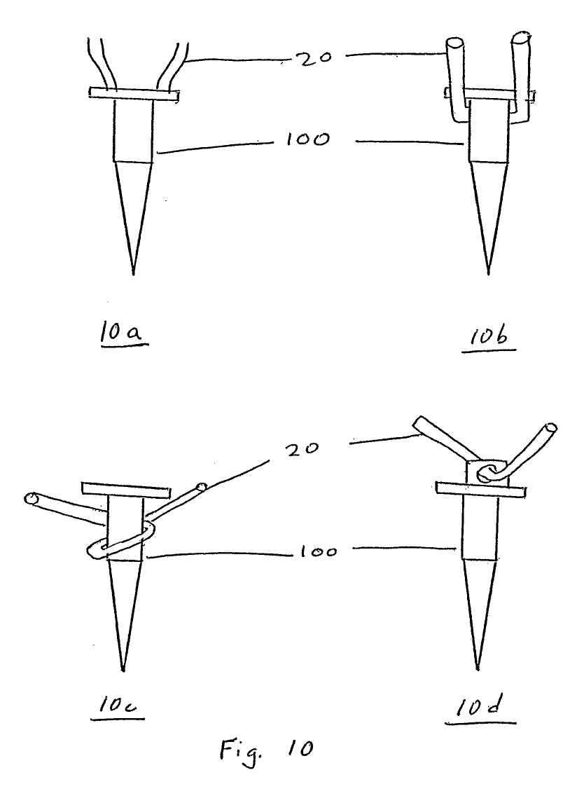

Alternatively, the surgical implant can utilize fasteners such as tacks to secure the mesh in place. Referring to FIGS. 10A-D, a variety of different tacks 100 can be used to secure the mesh 20 into place. The mesh 20 can be directly attached to the tack as seen in FIGS. 10A and 10D. Alternatively, the mesh 20 can be placed underneath the head of the tack prior to implantation as demonstrated by FIGS. 10B and 10C. Also, the edges of the mesh can be wrapped around a tack to further secure the two devices.

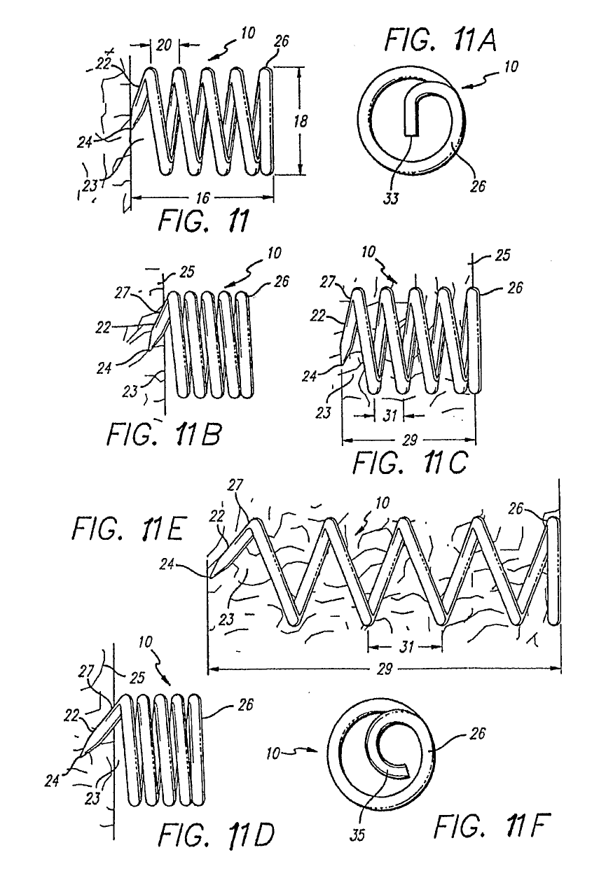

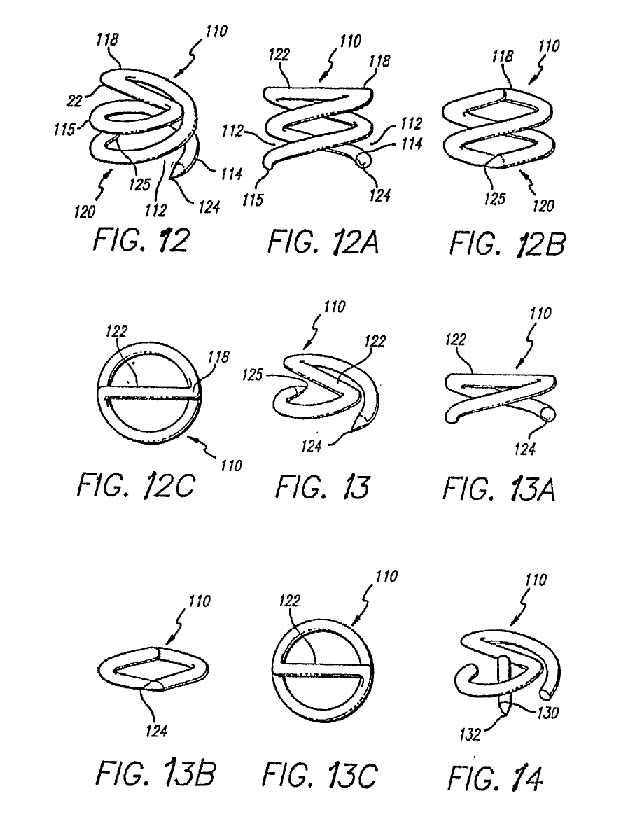

Configurations of additional fasteners which may be utilized to attach a wide-weave mesh of the present disclosure to tissue are helical fasteners depicted in FIG. 11 (including FIGS. 11A-F), FIG. 12 (including FIGS. 12A-C), FIG. 13 (including FIGS. 13A-C), and FIG. 14. The helical fasteners of FIGS. 11-14 correspond to FIGS. 1-4 of U.S. Pat. No. 6,562,051, the entire disclosure of which is incorporated by reference herein.

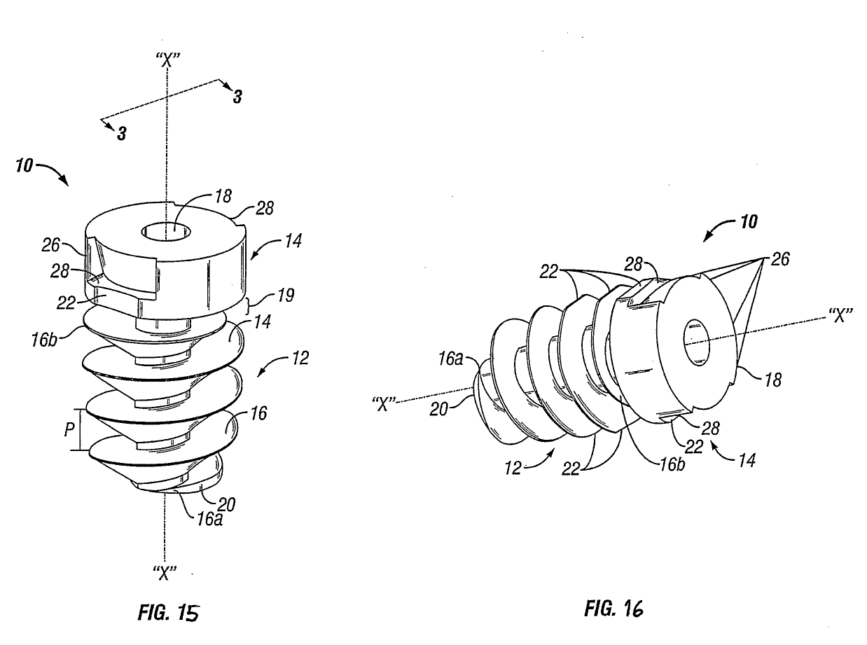

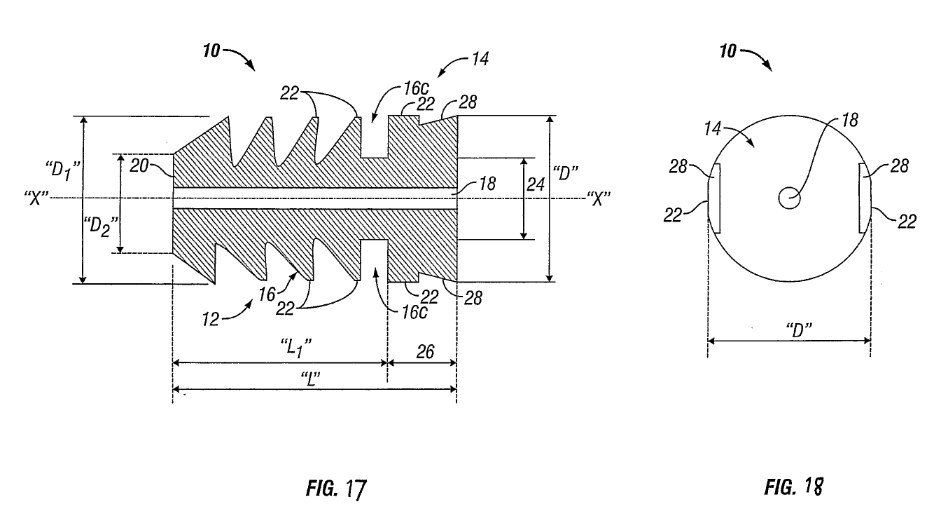

Other fasteners which may be utilized to attach a wide-weave mesh of the present disclosure to tissue are the screw fasteners depicted in FIGS. 15, 16, 17 and 18. The screw fasteners of FIGS. 15-18 correspond to FIGS. 1-4 of International Patent Application PCT US04/18702, filed on Jun. 14, 2004, the entire disclosure of which is incorporated by reference herein.

While the above description contains many specifics, these specifics should not be construed as limitations on the scope of the disclosure herein but merely as exemplifications of particularly useful embodiments thereof. Those skilled in the art will envision many other possibilities within the scope and spirit of the disclosure as defined by the claims appended hereto.

What is claimed is:

1. A wide-weave mesh comprising:
strands having a maximum residual mass density of from about 5 g/m² to about 50 g/m²;
spaces of about 1 mm to about 10 mm between the strands; and
a bioactive coating.

2. The wide-weave mesh of claim 1 wherein the bioactive coating comprises at least one bioactive agent.

3. The wide-weave mesh of claim 2 wherein at least one bioactive agent is selected from the group consisting of antimicrobial agents, antibacterial agents, anti-fungal agents, antibiotics, anti-viral agents, anti-tumor agents, anti-inflammatory agents, steroids, hormones, enzymes, analgesics, anesthetics, muscle relaxants, immunogenic agents, growth factors, immunosuppressants, lipids, lipopolysaccharides, polysaccharides, and peptides, polypeptides, proteins and combinations thereof.

4. The wide-weave mesh of claim 1 wherein the bioactive coating covers at least one side of the mesh.

5. The wide-weave mesh of claim 1 wherein the bioactive coating covers the entire mesh.

6. The wide-weave mesh of claim 1 wherein the bioactive coating comprises an absorbable material in combination with the at least one bioactive agent.

7. The wide-weave mesh of claim 6 wherein the bioactive coating degrades over a period of time from about 2 days to about 14 days.

8. The wide-weave mesh of claim 1 wherein the strands have a maximum residual mass density of from about 15 g/m² to about 40 g/m².

9. The wide-weave mesh of claim 1 wherein the strands have a diameter of from about 200 μm to about 600 μm.

10. The wide-weave mesh of claim 1 wherein the strands comprise at least one filament oriented to form pores in the strands.

11. The wide-weave mesh of claim 10 wherein the strands are formed from at least two filaments.

12. The wide-weave mesh of claim 10 wherein the filaments have a diameter of between about 0.02 mm to about 0.15 mm, and the pores in the strands have a diameter of from about 50 μm to about 200 μm in diameter.

13. The wide-weave mesh of claim 10 wherein the filaments have a diameter of about 0.08 mm to about 0.1 mm, and the pores in the strands have a diameter of from about 55 μm to about 75 μm.

14. The wide-weave mesh of claim 10 wherein the at least one filament comprises a synthetic material.

15. The wide-weave mesh of claim 14 wherein the at least one filament comprises polypropylene.

16. The wide-weave mesh of claim 10 wherein the at least one filament comprises an absorbable material.

17. The wide-weave mesh of claim 16 wherein the at least one filament comprises a polyester.

18. The wide-weave mesh of claim 1 further comprising rings of material which form pores in the mesh having a diameter of from about 50 μm to about 200 μm.

19. The wide-weave mesh of claim 1 wherein the strands of the mesh are comprised of bicomponent microfibers comprising a core material and a surface material.

20. The wide-weave mesh of claim 19 wherein the surface material comprises polylactic acid and the core material comprises polypropylene.

21. The wide-weave mesh of claim 1 wherein the strands comprise a material that has memory.

22. The wide-weave mesh of claim 1 wherein the mesh has a width from about 1 cm to about 10 cm and a length from about 1 cm to about 10 cm.

23. The wide-weave mesh of claim 1 wherein the mesh has a shape selected from the group consisting of round, circular, oval, ovoid, elliptical, and truncated elliptical.

24. The wide-weave mesh of claim 1 wherein the mesh has at least one circumferential member which extends along at least part of the perimeter of the mesh to provide a substantially smooth edge.

25. The wide-weave mesh of claim 24 wherein at least about 50% of the perimeter of the mesh is defined by the at least one circumferential member.

26. The wide-weave mesh of claim 24 wherein from about 80% to about 100% of the perimeter of the mesh is defined by the at least one circumferential member.

27. The wide-weave mesh of claim 24 wherein the mesh has a plurality of circumferential members arranged at different radial locations.

28. The wide-weave mesh of claim 27 wherein the circumferential members are arranged to join with one another in order to form an integral mesh.

29. The wide-weave mesh of claim 27 wherein the mesh further comprises transverse members which extend across the circumferential members thereby joining the circumferential members.

30. A method of treating uterovaginal prolapse comprising the steps of:
making an incision in the vaginal wall close to the opening of the vaginal cavity;
making a subcutaneous cut, through the incision, over and surrounding the area of the prolapse, which cut is substantially parallel to the vaginal wall; and
inserting a mesh according to claim 1, through the incision, into a space defined by the cut.

31. The method of treating uterovaginal prolapse of claim 30 wherein the incision in the vaginal wall is at the posterior extremity of the prolapse sac of the vaginal cavity.

32. The method of treating uterovaginal prolapse of claim 30 wherein the incision in the vaginal wall is at the anterior extremity of the prolapse sac of the vaginal cavity.

33. The method of treating uterovaginal prolapse of claim 30 wherein the mesh is attached to tissue utilizing a fastener selected from the group consisting of tacks, helical fasteners, screw fasteners, sutures, glues, staples, and clips.
